(19) United States
(12) Patent Application Publication
(10) Pub. No.: US 2024/0067624 A1
(43) Pub. Date: Feb. 29, 2024

(54) PROCESS FOR PREPARATION OF INSECTICIDAL ANTHRANILAMIDES

(71) Applicant: UPL LIMITED, Mumbai (IN)

(72) Inventors: Digish Manubhai Panchal, Mumbai (IN); Jigar Kantilal Desai, Mumbai (IN)

(21) Appl. No.: 18/260,567

(22) PCT Filed: Jan. 8, 2022
(86) PCT No.: PCT/IB2022/050124

§ 371 Date: Jul. 6, 2023

(30) Foreign Application Priority Data
Jan. 11, 2021 (IN) 202121001255

(51) Int. Cl. C07D 401/04; A01P 7/04

(52) U.S. Cl. CPC C07D 401/04; A01P 7/04

(57) ABSTRACT

Disclosed herein a convenient process for preparation of insecticidal anthranilamide compounds and intermediate thereof.

### FIELD OF THE INVENTION

The present invention relates to a convenient one pot process for preparation of insecticidal anthranilamides.

### BACKGROUND OF THE INVENTION

Anthranilamides are a new class of compounds with extremely potent insecticidal activity. These nitrogen-containing aromatic compounds selectively act on targeted ryanodine receptors which form calcium ion channels which are responsible for muscle function in insects.

Examples of insecticidal anthranilamides are cyantraniliprole, chlorantraniliprole, cyclaniliprole, tetrachlorantraniliprole and tetraniliprole. Chlorantraniliprole is a highly potent and selective activator of insect ryanodine receptor with exceptional activity on a broad range of Lepidoptera. It controls a wide range of chewing pests (primarily Lepidoptera, but also some Coleoptera, Diptera and Isoptera species) in a broad range of crops, including fruit, vegetables, vines, cotton, sugar cane, rice and grass.

US7232836 discloses preparation of chlorantraniliprole (E₁, Scheme 1) and US7247647 discloses preparation of cyantraniliprole (E₃, Scheme 2).

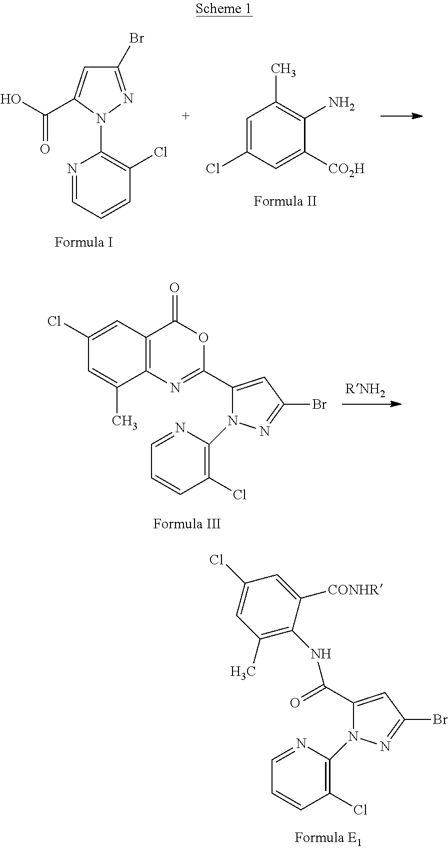

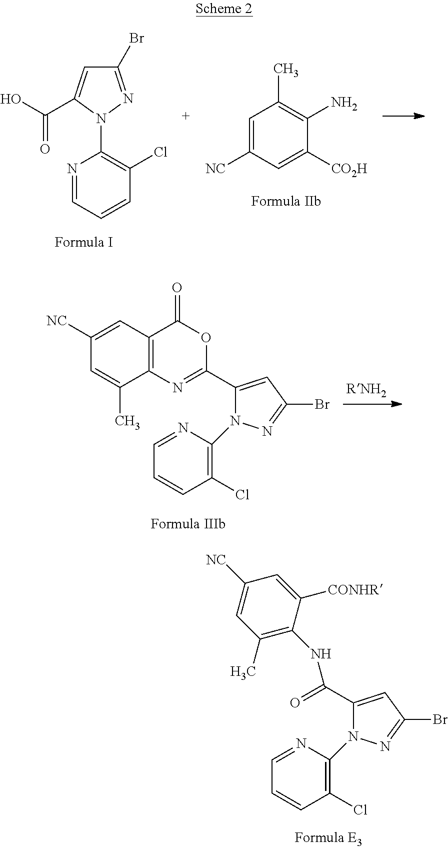

US8217179 discloses a process for preparation of anthranilamides. Inventors of the present invention noted that the reactions are carried out in hazardous solvents such benzene and toluene and using large volumes of solvent.

It has been observed that preparation of anthranilamides by known processes leads to formation of an undesired compound of Formula E₂ wherein X is chlorine or CN. It has been further noted that the formation compound of Formula B₂ (wherein Z is —OH or chlorine) in the previous steps of the synthesis leads to formation of compound of Formula E₂. Presence of compound of Formula E₂ and/or compound of Formula B₂ lead to the final product of poor quality which do not satisfy the regulatory requirements. Due to the structural similarity of the desired compounds of formula E₁/E₃ with compounds of Formula E₂, it is very difficult to separate those compounds.

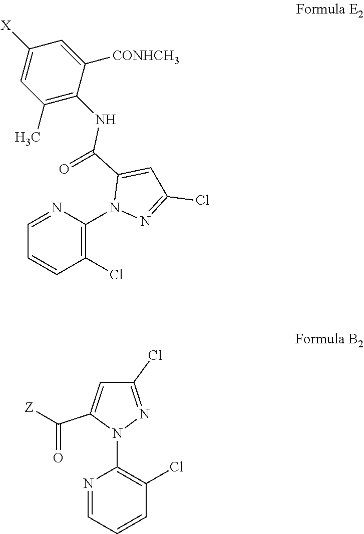

There is still a need for cost effective and improved production process for anthranilamide compounds that overcomes the above-mentioned drawbacks. Inventors of the present invention noted that carrying out the reactions in one pot using specific solvent system that minimizes side reactions, reduces environmental pollution and increases the process efficiency.

### OBJECTS OF THE INVENTION

It is an object of the present invention to provide a one pot process for preparation of anthranilamide compounds.

It is another object of the present invention to provide a convenient one pot process for the preparation of anthranilamide compound free from certain impurities.

It is another object of the present invention to provide an environmentally friendly and cost-effective process for the preparation of chlorantraniliprole free of certain impurities.

### SUMMARY OF THE INVENTION

In one aspect the present invention provides a process for the preparation of an anthranilamide compound of Formula E comprising reacting compound of Formula D with R′NH₂ wherein R′=a lower alkyl group or cycloalkyl to form anthranilamide compound of Formula E; wherein the reaction is carried out in a halogenated hydrocarbon solvent (Scheme 3).

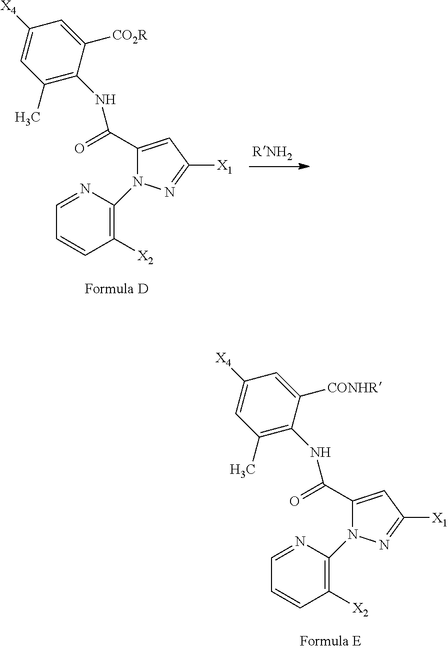

In another aspect, the present invention provides a one pot process for the preparation of an anthranilamide compound of Formula E comprising the steps of:
i) subjecting a compound of Formula A (3-Halo-1-(3-halo-2-pyridinyl)-4,5-dihydro-1H-pyrazole-5-carboxylic acid) to pyrazoline aromatization using a halogenating agent to form a compound of Formula B (3-Halo-1-(3-halo-2-pyridinyl)-1H-pyrazole-5-carbonyl halide) wherein X₁, X₂ and X₃ are halogens which are same or different;
ii) reacting the compound of Formula B with a compound of Formula C wherein X₄=halogen or CN and R=a lower alkyl group to form a compound of Formula D and
iii) reacting the compound of Formula D with a compound of formula R′NH₂ wherein R′=a lower alkyl group or cycloalkyl to form compound of Formula E
wherein the process is carried out in a halogenated hydrocarbon solvent (Scheme 3).

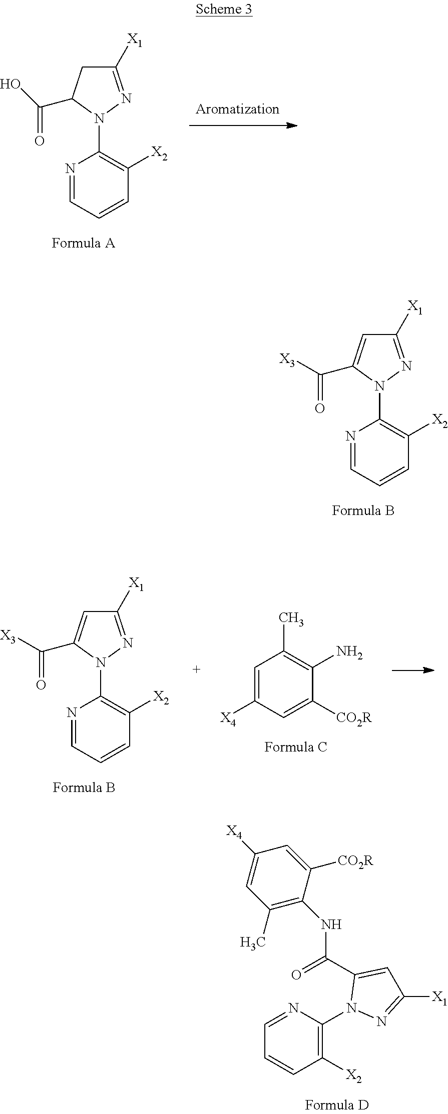

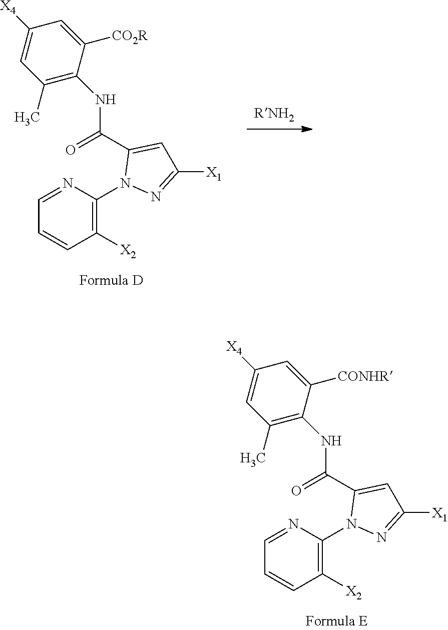

In another aspect the present invention provides a process for the preparation of a compound of Formula B (3-Halo-1-(3-halo-2-pyridinyl)-1H-pyrazole-5-carbonyl halide) said process comprising subjecting compound of Formula A (3-Halo-1-(3-halo-2-pyridinyl)-4,5-dihydro-1H-pyrazole-5-carboxylic acid) to pyrazoline aromatization using a halogenating agent in a halogenated hydrocarbon solvent wherein X₁, X₂ and X₃ are halogens which are same or different (Scheme 4).

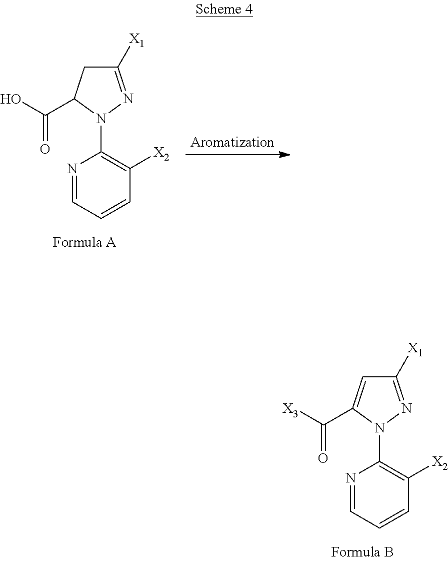

The present invention further provides a process for preparation of a compound of Formula B₁ (3-bromo-1-(3-chloro-2-pyridinyl)-1H-pyrazole-5-carbonyl chloride) said process comprising subjecting compound of Formula A₁ (3-Bromo-1-(3-chloro-2-pyridinyl)-4,5-dihydro-1H-pyrazole-5-carboxylic acid) to pyrazoline aromatization using a halogenating agent in a halogenated hydrocarbon solvent (Scheme 5).

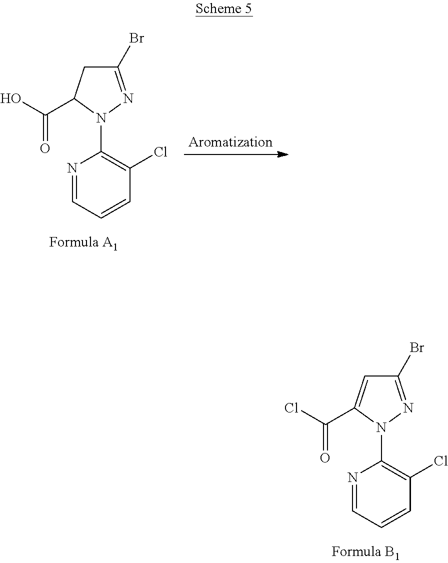 [SMILES: O=C(O)C1CC(Br)=NN1c1ncccc1Cl ; O=C(Cl)c1cc(Br)nn1-c1ncccc1Cl]

In another aspect the present invention provides chlorantraniliprole substantially free of a compound of formula E₂ wherein X is Cl.

In another aspect the present invention provides cyantraniliprole substantially free of a compound of formula E₂ wherein X is —CN.

A compound of Formula B₁ that is substantially free of a compound of formula B₂ wherein Z is —OH or chlorine.

### BRIEF DESCRIPTION OF DRAWING

FIG. 1 represents

Chromatogram of 3-Bromo-1-(3-chloro-2-pyridinyl)-1H-pyrazole-5-carbonyl chloride prepared using halogenated hydrocarbon solvent.

FIG. 2 represents Chromatogram of chlorantraniliprole prepared using halogenated hydrocarbon solvent.

FIG. 3 represents Chromatogram of 3-Bromo-1-(3-chloro-2-pyridinyl)-1H-pyrazole-5-carbonyl chloride prepared using toluene solvent.

### DETAILED DESCRIPTION

Those skilled in art will be aware that invention described herein is subject to variations and modifications other than those specifically described. It is to be understood that the invention described herein includes all such variations and modifications. The invention also includes all such steps, features and methods referred to or indicated in this specification, individually or collectively, and any and all combinations of any two or more said steps or features.

### Definitions

For convenience, before further description of the present invention, certain terms employed in the specification, examples are described here. These definitions should be read in light of the remainder of the disclosure and understood as by a person of skill in the art. Unless defined otherwise, all technical and scientific terms used herein have the same meaning as commonly understood by a person of ordinary skill in the art. The terms used throughout this specification are defined as follows, unless otherwise limited in specific instances.

The terms used herein are defined as follows.

As used in the specification and the claims, the singular forms “a”, “an”, and “the” include plural referents unless the context clearly dictates otherwise.

The present disclosure is not to be limited in scope by the specific embodiments described herein, which are intended for the purposes of exemplification only.

As used herein in this context, the expression “substantially free” will be understood to mean that 20% or less, 10% or less, 5% or less, 2% or less, or 1% or less or 0.5% or less of any known or unknown impurity as measured, for example, by HPLC.

The term “purity” means purity as determined by HPLC (“High Pressure Liquid Chromatography”).

The term “about” shall be interpreted to mean “approximately” or “reasonably close to” and any statistically insignificant variations therefrom.

As used herein, the terms “comprising” “including,” “having,” “containing,” “involving,” and the like are to be understood to be open-ended, i.e., to mean including but not limited to.

The terms “preferred” and “preferably” refer to embodiments of the invention that may afford certain benefits, under certain circumstances. In an embodiment, the aspects and embodiments described herein shall also be interpreted to replace the clause “comprising” with either “consisting of” or with “consisting essentially of” or with “consisting substantially of”.

As used herein, the term ‘lower alkyl’ refers to “(C₁-C₆) alkyl” which refers to the radical of saturated aliphatic groups, including straight or branched-chain alkyl groups. A straight-chain or branched chain alkyl has six or fewer carbon atoms in its backbone, for instance, C₁-C₆ for straight chain and C₃-C₆ for branched chain. As used herein, (C₁-C₆) alkyl refers to an alkyl group having from 1 to 6 carbon atoms. Representative examples of alkyl include, but are not limited to, methyl, ethyl, n-propyl, n-butyl, n-pentyl, n-hexyl, isopropyl, sec-butyl, isobutyl, tert-butyl, isopentyl, 2-methylbutyl and 3-methylbutyl.

As used herein, the term “halogen” refers to chlorine, fluorine, bromine or iodine.

The present invention provides a one pot process for preparation of anthranilamide compound of Formula E. The process avoids isolation of intermediates and each of the steps is carried out in a common solvent system. Using the present invention, it is possible to obtain anthranilamides compound of desired quality in good yield enabling the industrial operation of the process with a solvent selected from halogenated hydrocarbon solvents. It has been noted that the inventive process avoids the formation of undesired compound of Formula E₂ and/or compound of Formula B₂.

The present invention provides a one pot process for the preparation of an anthranilamide compound of Formula E comprising the steps of:
i) subjecting a compound of Formula A (3-Halo-1-(3-halo-2-pyridinyl)-4,5-dihydro-1H-pyrazole-5-carboxylic acid) to pyrazoline aromatization using a reagent system comprising a halogenating agent to form compound of Formula B (3-Halo-1-(3-halo-2-pyridinyl)-1H-pyrazole-5-carbonyl halide) wherein X₁, X₂ and X₃ are halogens which are same or different;
ii) reacting compound of Formula B with a compound of Formula C wherein X₄=halogen or CN and R=a lower alkyl group to form a compound of Formula D; and
iii) reacting compound of Formula D with a compound of formula R′NH₂ wherein R′=a lower alkyl group or cycloalkyl to form anthranilamide compound of Formula E.

In an embodiment each step of the process is carried out in a halogenated hydrocarbon solvent. In an embodiment the process for preparation of compound E is represented in below Scheme 3.

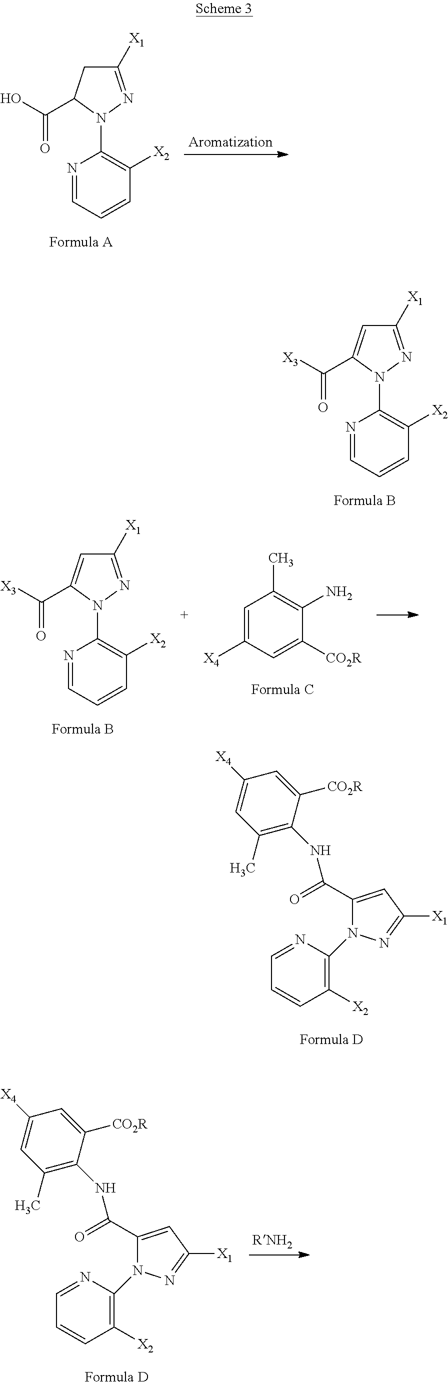

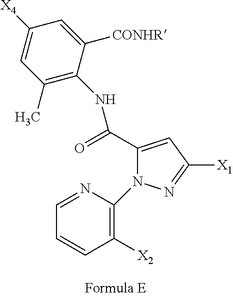

The product from each step may be isolated at the end of the step or, more=preferably, the reaction may be proceeded without isolation or purification until the last step.

In an embodiment the compound of Formula B (3-Halo-1-(3-halo-2-pyridinyl)-1H-pyrazole-5-carbonyl halide) is prepared by subjecting the compound of Formula A (3-Halo-1-(3-halo-2-pyridinyl)-4,5-dihydro-1H-pyrazole-5-carboxylic acid) to pyrazoline aromatization using a halogenating agent to form a compound of Formula B wherein X₁, X₂ and X₃ is independently a halogen which is same or different.

With respect to the present invention, pyrazoline aromatization refers to reaction in which 3-Halo-1-(3-halo-2-pyridinyl)-4,5-dihydro-1H-pyrazole-5-carboxylic acid is converted to the corresponding 3-Halo-1-(3-halo-2-pyridinyl)-1H-pyrazole-5-carbonyl halide.

In one preferred embodiment the compound of Formula B (3-Halo-1-(3-halo-2-pyridinyl)-1H-pyrazole-5-carbonyl halide) is prepared by subjecting the compound of Formula A (3-Halo-1-(3-halo-2-pyridinyl)-4,5-dihydro-1H-pyrazole-5-carboxylic acid) to pyrazoline aromatization using a halogenating agent to form a compound of Formula B wherein X₁, X₂ and X₃ is selected from bromine and chlorine.

In an embodiment the halogenating agent is selected from thionyl chloride, phosgene, oxalyl chloride, phosphorus oxychloride, phosphorus trichloride or phosphorus pentachloride.

In a preferred embodiment the halogenating agent is thionyl chloride.

In an embodiment the molar ratio of pyrazoline to the halogenating agent is from about 1:2 to about 1:5.

In an embodiment the pyrazoline aromatization is carried out optionally in the presence of a catalyst.

In an embodiment the catalyst is an organic base.

In an embodiment the catalyst is triethylamine or dimethylformamide.

In an embodiment the reaction is carried out at a temperature of 30°-70° C., preferably at a temperature of 30°-65° C.

In an embodiment the halogenated hydrocarbon solvent is selected from dichloromethane, dichloroethane, trichloromethane, chloroform, carbon tetrachloride and halogenated aromatic hydrocarbons.

In an embodiment Formula A (3-Halo-1-(3-halo-2-pyridinyl)-4,5-dihydro-1H-pyrazole-5-carboxylic acid) represents a compound wherein X₁ is bromine and X₂ is chlorine.

In an embodiment Formula B (3-Halo-1-(3-halo-2-pyridinyl)-1H-pyrazole-5-carbonyl halide) represents a compound wherein X₁ is bromine and X₂ and X₃ are chlorine.

In an embodiment the compound of Formula B is not isolated or purified.

In an embodiment the present process provides a compound of Formula B substantially free of a compound of Formula B₂.

In the present process the compound of Formula B is reacted with a compound of Formula C wherein X₄=halogen and R=a lower alkyl group to form a compound of Formula D.

In an embodiment the compound of Formula B is reacted with a compound of Formula C wherein X₄=—CN and R=a lower alkyl group to form a compound of Formula D.

In an embodiment the compound of Formula B is reacted with a compound of Formula C in a halogenated hydrocarbon solvent.

In an embodiment the halogenated hydrocarbon solvent is selected from dichloromethane, dichloroethane, trichloromethane, chloroform, carbon tetrachloride and halogenated aromatic hydrocarbons.

In an embodiment the compound of Formula B is reacted with a compound of Formula C optionally in the presence of a base.

In an embodiment the base used is a weak inorganic base.

In an embodiment the base used can be alkali or alkaline earth metal salts.

In an embodiment the base such as sodium carbonate or potassium carbonate can be used in the process.

In an embodiment Formula C represents a compound wherein X₄=Chlorine and R=Methyl group.

In an embodiment Formula C represents a compound wherein X₄=Chlorine and R=Ethyl group.

In an embodiment Formula C represents a compound wherein X₄=—CN and R=Methyl group.

In an embodiment Formula C represents a compound wherein X₄=—CN and R=Ethyl group.

In an embodiment Formula D represents a compound wherein X₁=Bromine, X₂, X₄=Chlorine and R=Methyl group.

In an embodiment Formula D represents a compound wherein X₁=Bromine, X₂, X₄=Chlorine and R=Ethyl group.

In an embodiment Formula D represents a compound wherein X₁=Bromine, X₂=Chlorine, X₄=—CN and R=Methyl group.

In an embodiment Formula D represents a compound wherein X₁=Bromine, X₂=Chlorine, X₄=—CN and R=Ethyl group.

In further embodiment the compound of Formula D is reacted with compound of formula R′NH₂ wherein R′=a lower alkyl group to form anthranilamide compound of Formula E.

In an embodiment Formula E represent a compound wherein X₁=Bromine, X₂, X₄=Chlorine and R′=Methyl.

In an embodiment Formula E represent a compound wherein X₁=Bromine, X₂=Chlorine, X₄=—CN and R′=-Methyl.

In an embodiment, the reaction of compound of Formula D with a compound of formula R′NH₂ is carried out by purging methyl amine gas.

In an embodiment, the reaction is carried out by using methyl amine aqueous solution.

In an embodiment the present invention provides a one pot process for the preparation of chlorantraniliprole of Formula E₁ said process comprising:
reacting compound of Formula D₁ with methyl amine in a halogenated hydrocarbon solvent to form chlorantraniliprole of Formula E₁.

In an embodiment the present invention provides a one pot process for the preparation of chlorantraniliprole of Formula E₁ said process comprising the steps of:
i) subjecting compound of Formula A₁ to pyrazoline aromatization using a halogenating agent to form compound of Formula B₁;
ii) reacting compound of Formula B₁ with a compound of Formula C₁ to form a compound of Formula D₁; and
iii) reacting compound of Formula D₁ with methyl amine to form chlorantraniliprole of Formula E₁ wherein each of the steps is carried out in a halogenated hydrocarbon solvent (Scheme 6).

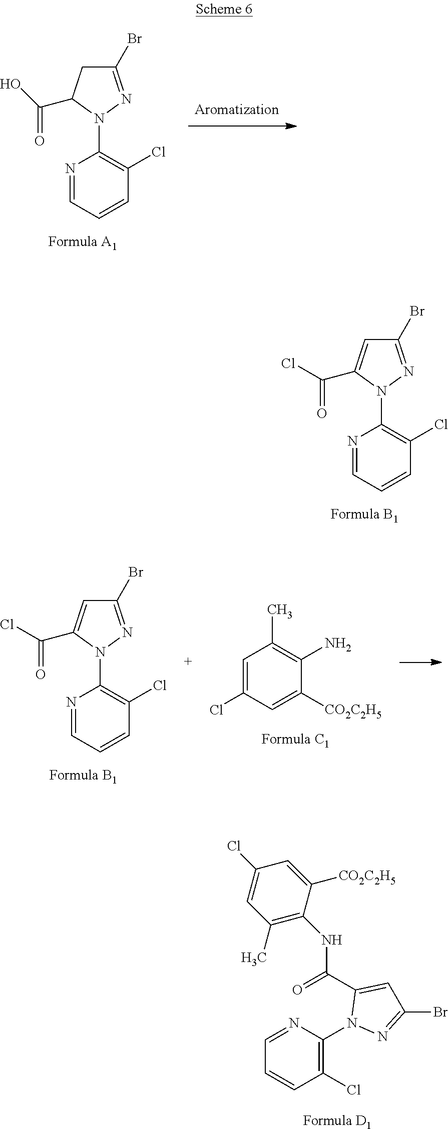 [SMILES: O=C(O)C1CC(Br)=NN1c1ncccc1Cl ; O=C(Cl)c1cc(Br)nn1-c1ncccc1Cl ; O=C(Cl)c1cc(Br)nn1-c1ncccc1Cl ; CCOC(=O)c1cc(Cl)cc(C)c1N ; CCOC(=O)c1cc(Cl)cc(C)c1NC(=O)c1cc(Br)nn1-c1ncccc1Cl]

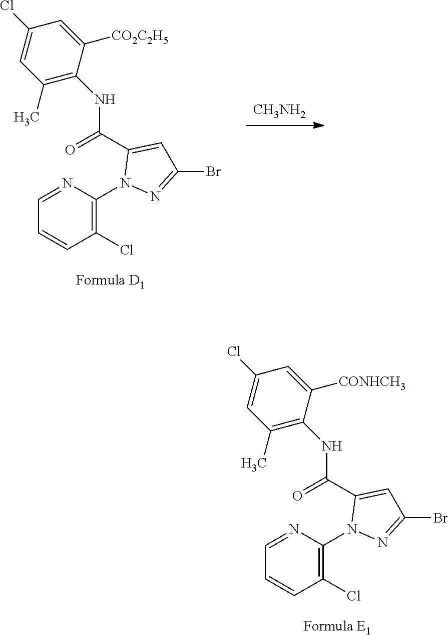

In an embodiment the present invention provides chlorantraniliprole substantially free of a compound of formula E₂.

In an embodiment the present invention provides chlorantraniliprole that contains less than 1.0% by weight of a compound of formula E₂.

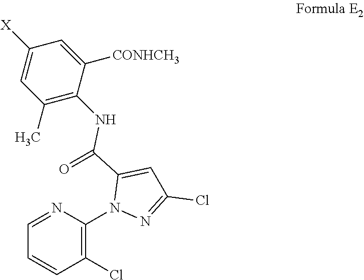

In an embodiment the present invention provides chlorantraniliprole substantially free of a compound of formula B₂ wherein Z is —OH or chlorine.

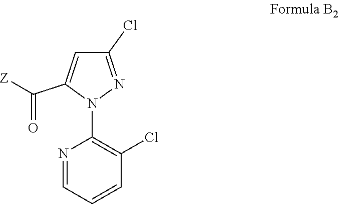

In an embodiment the present invention provides chlorantraniliprole substantially free of a compound of formula B₃ ((3-chloro-1-(3-chloro-2-pyridinyl)-1H-pyrazole-5-carbonyl chloride).

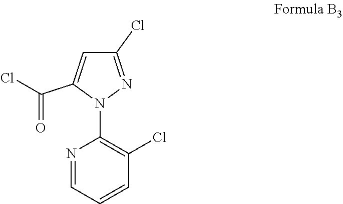 [SMILES: O=C(Cl)c1cc(Cl)nn1-c1ncccc1Cl]

In an embodiment the present invention provides chlorantraniliprole that contains less than 1.0% by weight of a compound of formula B₃.

In an embodiment the present invention provides cyantraniliprole that contains less than 1.0% by weight of a compound of formula B₃.

In an embodiment the present invention provides a one pot process for the preparation of cyantraniliprole of Formula E₃ said process comprising: reacting compound of Formula D₂ with methyl amine in a halogenated hydrocarbon solvent to form cyantraniliprole of Formula E₃.

In an embodiment the present invention provides a one pot process for the preparation of cyantraniliprole of Formula E₃ said process comprising the steps of:
i) subjecting compound of Formula A₁ to pyrazoline aromatization using a halogenating agent to form compound of Formula B₁;
ii) reacting compound of Formula B₁ with a compound of Formula C₂ to form a compound of Formula D₂; and
iii) reacting compound of Formula D₂ with methyl amine to form cyantraniliprole of Formula E₃ wherein each of the steps is carried out in a halogenated hydrocarbon solvent (Scheme 7).

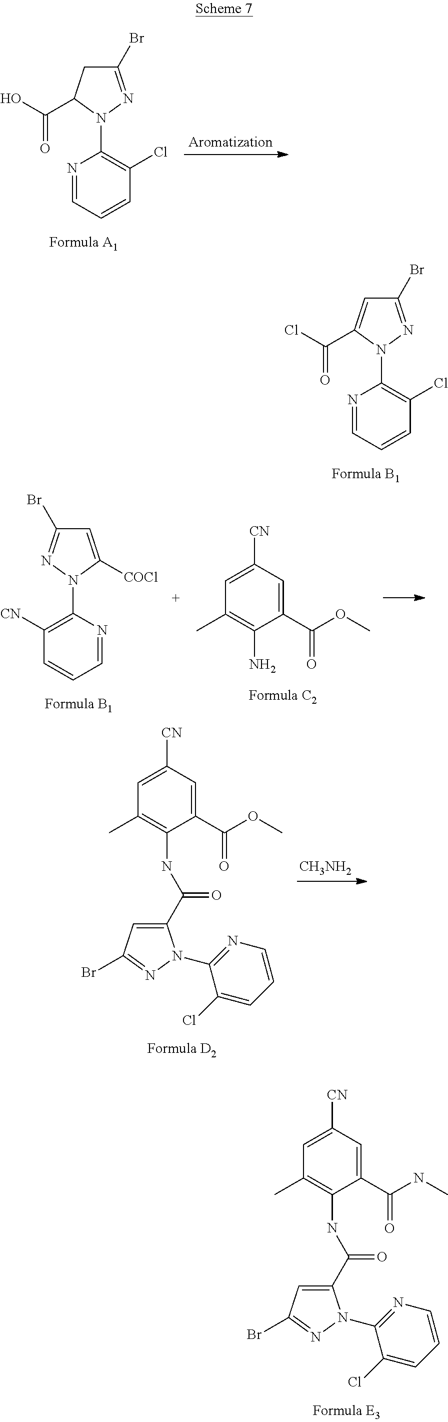

In an embodiment the present invention provides cyantraniliprole substantially free of a compound of formula E₄.

In an embodiment the present invention provides cyantraniliprole that contains less than 1.0% by weight of a compound of formula E₄.

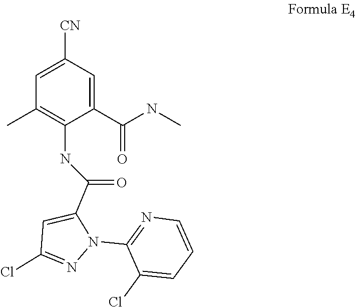 [SMILES: CNC(=O)c1cc(C#N)cc(C)c1NC(=O)c1cc(Cl)nn1-c1ncccc1Cl]

The present invention provides a process for the preparation of a compound of Formula B (3-Halo-1-(3-halo-2-pyridinyl)-1H-pyrazole-5-carbonyl halide) said process comprising subjecting compound of Formula A (3-Halo-1-(3-halo-2-pyridinyl)-4,5-dihydro-1H-pyrazole-5-carboxylic acid) to pyrazoline aromatization using a halogenating agent in a halogenated hydrocarbon solvent wherein X₁, X₂ and X₃ are halogens which are same or different (Scheme 4).

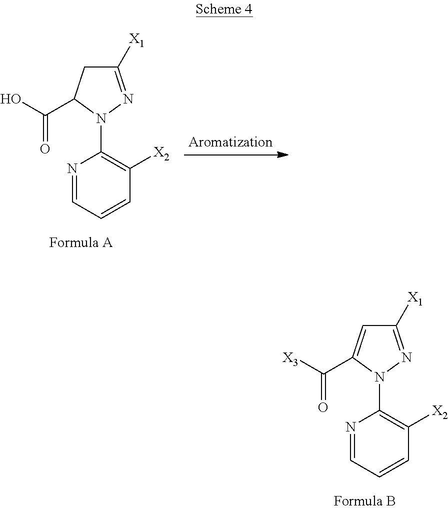

The present invention further provides a process for preparation a compound of Formula B₁ (3-bromo-1-(3-chloro-2-pyridinyl)-1H-pyrazole-5-carbonyl chloride) said process comprising subjecting compound of Formula A₁ (3-Bromo-1-(3-chloro-2-pyridinyl)-4,5-dihydro-1H-pyrazole-5-carboxylic acid) to pyrazoline aromatization using a halogenating agent in a halogenated hydrocarbon solvent (Scheme 5).

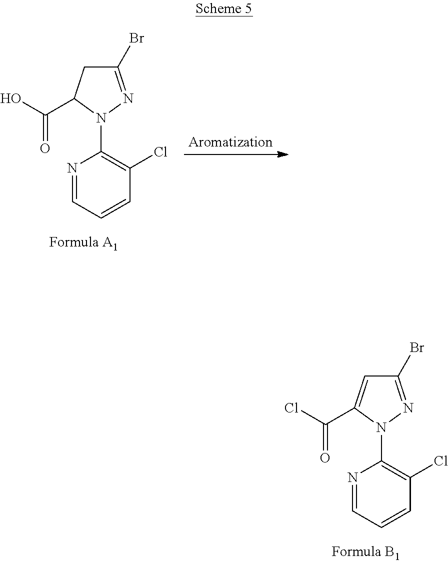 [SMILES: O=C(O)C1CC(Br)=NN1c1ncccc1Cl ; O=C(Cl)c1cc(Br)nn1-c1ncccc1Cl]

In an embodiment the present invention provides a compound of Formula B₁ that is substantially free of a compound of formula B₂.

In an embodiment the present invention provides a compound of Formula B₁ that contains less than 1.0% by weight of a compound of formula B₃.

In an embodiment the present invention provides a process for preparation of compound of Formula B₁ in conditions that are not favourable for the formation of a compound of formula B₂.

In an embodiment the halogenating agent is selected from thionyl chloride, phosgene, oxalyl chloride, phosphorus oxychloride, phosphorus trichloride, phosphorus tribromide, phosphorus pentachloride.

In a preferred embodiment the halogenating agent is thionyl chloride.

In an embodiment the molar ratio of pyrazoline to halogenating agent is from about 1:2 to about 1:5.

In an embodiment the pyrazoline aromatization is carried out optionally in the presence of a catalyst.

In an embodiment the catalyst is an organic base.

In an embodiment the catalyst is triethylamine or dimethylformamide.

In an embodiment the reaction is carried out at a temperature of 30°-70° C., preferably at a temperature of 30°-65° C.

In an embodiment halogenated hydrocarbon solvent is selected from dichloromethane, dichloroethane, trichloromethane, chloroform, carbon tetrachloride and halogenated aromatic hydrocarbons.

In an embodiment the halogenated hydrocarbon solvent is dichloromethane.

In an embodiment Formula B (3-Halo-1-(3-halo-2-pyridinyl)-1H-pyrazole-5-carbonyl halide) according to the present invention that is substantially free of a compound of formula B2 may be used for preparing high purity insecticidal anthranilamides.

In an embodiment compound of Formula A₁ (3-Bromo-1-(3-chloro-2-pyridinyl)-4,5-dihydro-1H-pyrazole-5-carboxylic acid) is prepared by processes known in the art.

In an embodiment 3 compound of Formula A₁ is prepared by treating ethyl 2-(3-chloro-2-pyridinyl)-5-oxo-3-pyrazolidinecarboxylate with phosphorous oxybromide to obtain ethyl 3-Bromo-1-(3-chloro-2-pyridinyl)-4,5-dihydro-1H-pyrazole-5-carboxylate followed by hydrolysis.

In an embodiment hydrolysis of ethyl 3-Bromo-1-(3-chloro-2-pyridinyl)-4,5-dihydro-1H-pyrazole-5-carboxylate is performed by an alkali optionally in presence of a solvent.

In an embodiment the present invention provides an insecticidal composition comprising chlorantraniliprole or cyantraniliprole produced according to present inventive process as described herein and agrochemically acceptable excipients.

In an embodiment the present invention provides a method of controlling insects comprising applying to the pests or to their locus an insecticidally effective amount of chlorantraniliprole or cyantraniliprole produced according to present inventive process as described herein.

### Advantages of the Present Invention

a) the reaction is carried out in one pot without isolation of intermediates;
b) the reaction is carried out in a halogenated hydrocarbon solvent system;
c) the reaction condition allows the preparation of anthranilamides that are free of certain impurities which are difficult to eliminate from the final reaction mass;
d) the reaction leads to the formation of anthranilamides with high purity required to meet regulatory standards; and
e) one pot process and single solvent system makes the process cost effective and has reduction in effluent.

Further advantages and other parameters of the present invention is illustrated by the below given examples. However, the scope of the present invention is not limited by the examples in any manner. It will be appreciated by any person skilled in this art that the present invention includes aforesaid examples and further can be modified and altered within the technical scope of the present invention.

### EXAMPLES

### Example 1

### Preparation of 3-Bromo-1-(3-chloro-2-pyridinyl)-1H-pyrazole-5-carbonyl chloride (B₁)

Ethyl 2-(3-chloro-2-pyridinyl)-5-oxo-3-pyrazolidinecarboxylate (1.27 Kg) and acetonitrile (4.45 Kg) were charged in a reaction kettle. Phosphorus oxybromide (0.941 Kg) was added to the mixture dropwise at 25-30° C. After the complete addition, the reaction mass was stirred at 68-70° C. for 2 to 3 hours for completion of the reaction. Acetonitrile was then recovered, and the reaction mass was cooled to 30° C. Water (6.2 Kg) was added to the mass and the pH was adjusted between 7.5 to 8 using 20% sodium carbonate solution (3.1 Kg). Dichloromethane (7.2 Kg) was then added and stirred, and the layers were separated. The organic layer was washed with water, dried and solvent was evaporated to get ethyl 3-bromo-1-(3-chloro-2-pyridinyl)-4,5-dihydro-1H-pyrazole-5-carboxylate (1.544 Kg; Purity 97% by HPLC).

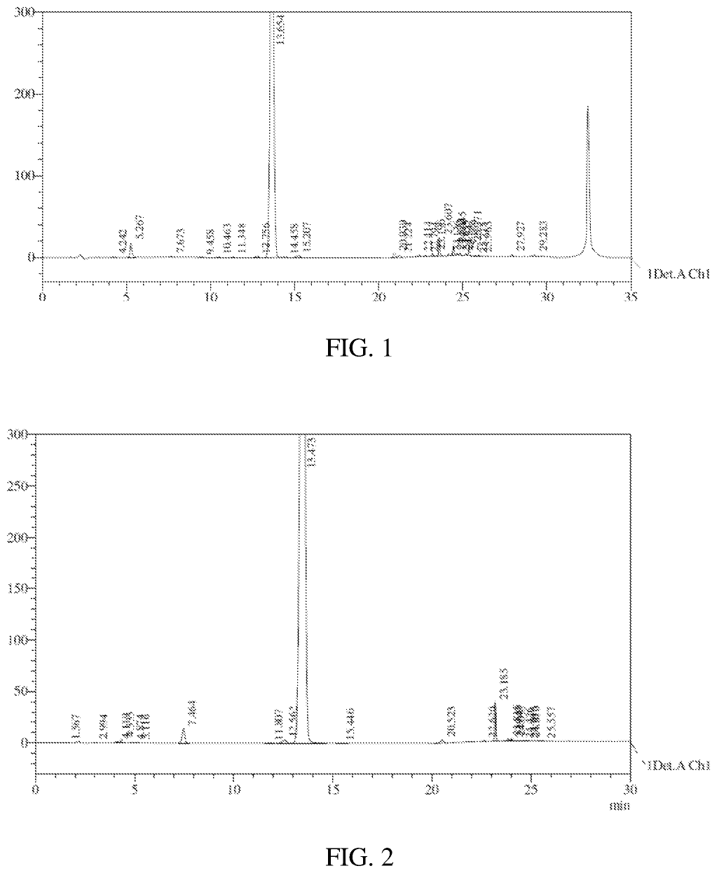

Ethyl 3-bromo-1-(3-chloro-2-pyridinyl)-4,5-dihydro-1H-pyrazole-5-carboxylate (1.544 Kg) was charged in a reaction kettle. A solution of dilute caustic lye (water 4.64 Kg, caustic lye 0.467 Kg) was added at 35-40° C. The reaction mass was then stirred for 3 hours. After completion of the reaction, ethanol was recovered from the reaction mass and dichloromethane (7.416 Kg) and water (0.620 Kg) were added at 25-30° C. and the mixture was stirred for 1 hour at the same temperature. pH of reaction mass was then adjusted to 2.0 to 2.5 using 10% hydrochloric acid (1.730 Kg). Organic layer was separated, washed and dried to get the solution of 3-Bromo-1-(3-chloro-2-pyridinyl)-4,5-dihydro-1H-pyrazole-5-carboxylic acid (8.598 Kg −1.300 Kg in 7.384 Kg of dichloromethane). Dichloromethane (0.86 Kg) and DMF (0.0040 Kg) were added to the solution at 25-30° C. under nitrogen atmosphere. A solution of thionyl chloride (1.17 Kg) in dichloromethane (1.17 Kg) was added dropwise to the reaction mass at 35-40° C. and then maintained for 2-3 hours at 38-40° C. After completion of the reaction, thionyl chloride and dichloromethane were recovered completely. A 25% solution of 3-bromo-1-(3-chloro-2-pyridinyl)-1H-pyrazole-5-carbonyl chloride was prepared by addition of dichloroethane (4.800 Kg) (HPLC analysis: B₁: 92%; compound of formula B₃: 0.30%; FIG. 1).

### Example 2

### Preparation of 2-[(3-bromo-1-(3-chloro-2-pyridinyl)-1H-pyrazol-5-yl]carbonyl]amino]-5-chloro-3-methyl-, Benzoic acid, methyl ester

A reaction kettle was charged with 21% solution of methyl 2-amino-5-chloro-3-methylbenzoate in dichloroethane (2.538 Kg). The reaction mass was heated to 65-70° C. within 1 hr. A 25% solution of 3-bromo-1-(3-chloro-2-pyridinyl)-1H-pyrazole-5-carbonyl chloride in dichloroethane (4.7 Kg) was added to the mixture in 5-6 hours under nitrogen atmosphere and stirred until completion of the reaction. The reaction mass was cooled to 40-50° C. Water (1.32 Kg) was then added to the reaction mass and organic layer was separated from the aqueous layer. The organic layer was washed with dilute caustic solution (1.32 Kg of 1% solution) and finally with water (1.32 Kg). Solvent was evaporated from the organic layer partially until a 15% solution of 2-[(3-bromo-1-(3-chloro-2-pyridinyl)-1H-pyrazol-5-yl]carbonyl]amino]-5-chloro-3-methyl-, Benzoic acid, methyl ester (8.530 Kg) was obtained (yield=96% and purity=94%).

### Example 3 Preparation of Chlorantraniliprole (E₁)

A reaction kettle was charged with a 15% solution of 2-[(3-bromo-1-(3-chloro-2-pyridinyl)-1H-pyrazol-5-yl]carbonyl]amino]-5-chloro-3-methyl-, Benzoic acid, methyl ester in dichloroethane (8.530 Kg). The reaction mass was cooled to 15-20° C. and then purged with methyl amine gas (0.700 Kg) for about 5-7 hours (at a rate of 0.100 to 0.150 Kg/Hr). The mass was then purged with nitrogen for 1-2 hours and the solvent was evaporated partially. The product was then filtered and washed with acetonitrile (1.27 Kg). A slurry of the wet cake was prepared in water (4.57 Kg) at 45-50° C. and stirred for 1 hour. The product was filtered and washed with water (1.5 Kg) to get Chlorantraniliprole (E₁: 1.00 Kg; purity: 96.8%; compound of formula B₃: 0.053; compound of formula E₂: 0.295%; FIG. 2).

### Example 4

### Comparative Example: Preparation of 3-Bromo-1-(3-chloro-2-pyridinyl)-1H-pyrazole-5-carbonyl chloride (B₁ ) using toluene as solvent

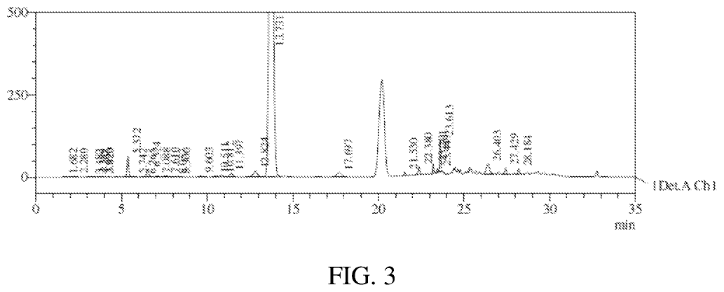

A reaction kettle was charged with a solution of 3-Bromo-1-(3-chloro-2-pyridinyl)-4,5-dihydro-1H-pyrazole-5-carboxylic acid in toluene (8.5 Kg -1.300 Kg in 7.24 Kg of toluene). Toluene (0.86 Kg) and DMF (0.0040 Kg) were further added to the solution at 25-30° C. under nitrogen atmosphere. A solution of thionyl chloride (1.17 Kg) in toluene (1.17 Kg) was added dropwise to the reaction mass at 25-30° C. and the reaction mass was then heated to reflux for 2-3 hours. After the completion of the reaction, the reaction mass was concentrated under vacuum to get the product (4.800 Kg; Purity: 88.7%; compound of formula B₃: 1.13%, FIG. 3).

### Example 5

### Preparation of 3-Bromo-1-(3-chloro-2-pyridinyl)-1H-pyrazole-5-carbonyl chloride (B₁)

Ethyl 2-(3-chloro-2-pyridinyl)-5-oxo-3-pyrazolidinecarboxylate (100 g) and acetonitrile (350 g) were charged in a reaction kettle. Phosphorus oxybromide (74.1 g) was added to the mixture dropwise at 25-30° C. After the addition was complete, the reaction mass was stirred at 68-70° C. for 2 to 3 hours for completion of the reaction. Acetonitrile was then recovered, and the reaction mass was cooled to 30°. Water (488 g) was added to the mass and the pH was adjusted to 8.5 to 8.9 using 20% sodium carbonate solution (244 g). Dichloromethane was then added and stirred and layers were separated. The organic layer was washed with water, dried and solvent evaporated to get ethyl 3-bromo-1-(3-chloro-2-pyridinyl)-4,5-dihydro-1H-pyrazole-5-carboxylate (121.6 g; Purity 98.1% by HPLC).

Ethyl3-bromo-1-(3-chloro-2-pyridinyl)-4,5-dihydro-1H-pyrazole-5-carboxylate (121.6 g) and 365.5 gm Water were charged in a reaction kettle. A solution of dilute caustic lye (36.8 g) was added at 35-40° C. The reaction mass was then stirred for 3 hours. After completion of the reaction, ethanol was recovered from the reaction mass and dichloromethane (584 g) and water were added at 25-30° C. and the mixture was stirred for 1 hour at the same temperature. pH of reaction mass was then adjusted to 2.0 to 2.5 using 10% hydrochloric acid (136 g). Organic layer was separated, washed and dried to get the solution of 3-Bromo-1-(3-chloro-2-pyridinyl)-4,5-dihydro-1H-pyrazole-5-carboxylic acid (677 g) DMF (0.315 g) was added to the solution at 25-30° C. under nitrogen atmosphere. A solution of thionyl chloride (92 g) in dichloromethane (92 g) was added dropwise to the reaction mass at 35-40° C. and then maintained for 2-3 hours at 38-40° C. After completion of the reaction, thionyl chloride and dichloromethane were recovered completely. A 25% solution of 3-bromo-1-(3-chloro-2-pyridinyl)-1H-pyrazole-5-carbonyl chloride (115 g) was prepared by addition of dichloroethane (263 g) (HPLC analysis: B₁: 91.9%; compound of formula B₃: 0.29%).

### Example 6: Preparation of Chlorantraniliprole (E₁)

A reaction kettle was charged with a solution of methyl 2-amino-5-chloro-3-methylbenzoate in dichloroethane (140 g). The reaction mass was heated to 65-70° C. within 1 hr. A solution of 3-bromo-1-(3-chloro-2-pyridinyl)-1H-pyrazole-5-carbonyl chloride in dichloroethane (212 g) was added to the mixture in 3-4 hours under nitrogen atmosphere and stirred until completion of the reaction. The reaction mass was cooled to 40-50° C. Water (91 g) was then added to the reaction mass and organic layer was separated from the aqueous layer. The organic layer was washed with dilute caustic solution (1% solution) and finally with water (91 g). Solvent was evaporated from the organic layer partially until a 15% solution of 2-[(3-bromo-1-(3-chloro-2-pyridinyl)-1H-pyrazol-5-yl]carbonyl]amino]-5-chloro-3-methyl-, Benzoic acid, methyl ester (464 g, 15% solution) was obtained (yield=96%). The reaction mass was cooled to 15-20° C. and then purged with methyl amine gas (38 g) for about 4-5 hours (at a rate of 0.100 to 0.150 Kg/Hr). The mass was then purged with nitrogen for 1-2 hours and the solvent was evaporated partially. The product was then filtered and washed with acetonitrile (70 g). A slurry of the wet cake was prepared in water (242 g) at 45-50° C. and stirred for 1 hour. The product was filtered and washed with water (90 g) to get Chlorantraniliprole (E₁: 54.3g; purity: 96.6%; compound of formula B₃: 0.11%; compound of formula E₂: 0.28%)

### Example 7: Preparation of 2-[(3-bromo-1-(3-chloro-2-pyridinyl)-1H-pyrazol-5-yl]carbonyl]amino]-5-cyano-3-methyl-, Benzoic acid, methyl ester

Dichloroethane (870 gm) and methyl 2-amino-5-cyano-3-methylbenzoate (85.9 g) were charged into a reaction kettle. The reaction mass was heated to 65-70° C. within 1 hr. A 25% solution of 3-bromo-1-(3-chloro-2-pyridinyl)-1H-pyrazole-5-carbonyl chloride in dichloroethane (390 g in 191 gm of dichloroethane) was added to the mixture in 5-6 hours under nitrogen atmosphere and stirred until completion of the reaction. Dichloroethane was recovered from the reaction mass and acetonitrile (260 g) was added at 40-50° C. and the mixture was stirred for 1 hour at the same temperature then cooled to 25-30° C. The product was filtered and washed with acetonitrile. A slurry of the wet cake was prepared in 5% Sodium carbonate solution (261 g) at 25-30° C. and stirred for 1 hour then filtered and washed with water (174 g) to get 2-[(3-bromo-1-(3-chloro-2-pyridinyl)-1H-pyrazol-5-yl]carbonyl]amino]-5-cyano-3-methyl-, Benzoic acid, methyl ester (160 g).

### Example 8: Preparation of Cyantraniliprole (E₃)

Dichloroethane (1072 gm) and 2-[(3-bromo-1-(3-chloro-2-pyridinyl)-1H-pyrazol-5-yl]carbonyl]amino]-5-cyano-3-methyl-, Benzoic acid, methyl ester (160 g) were charged into a reaction kettle. The reaction mass was cooled to 15-20° C. and then purged with methyl amine gas (66 g) for about 5-7 hours (at a rate of 0.100 to 0.150 Kg/Hr). The mass was then purged with nitrogen for 1-2 hours and dichloroethane was recovered from the reaction mass and acetonitrile (400 g) was added at 40-50° C. and the mixture was stirred for 1 hour at the same temperature. The reaction mass was cooled to 25-30° C. The product was then filtered and washed with acetonitrile and water to get Cyantraniliprole (102 g; purity: 97.56% compound of formula E₄: 0.45%).

1. A process for the preparation of an anthranilamide compound of Formula E, said process comprising:
i) subjecting a compound of Formula A to pyrazoline aromatization by reacting with a halogenating agent to form a compound of Formula B, wherein X₁, X₂ and X₃ are halogens, which are X₁, X₂ and X₃ are the same or different;
ii) reacting the compound of Formula B with a compound of Formula C, wherein X₄ is halogen or —CN and R is a lower alkyl group, to form a compound of Formula D; and
iii) reacting the compound of Formula D with a compound of formula R′NH₂, wherein R′ is a lower alkyl group or cycloalkyl, to form the anthranilamide compound of Formula E;
wherein each step of the process i), ii) and iii) is carried out in a halogenated hydrocarbon solvent

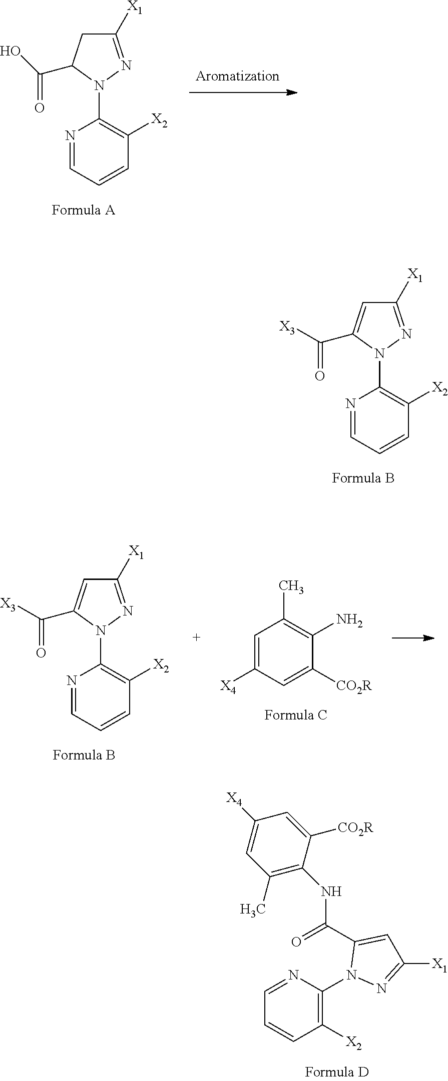

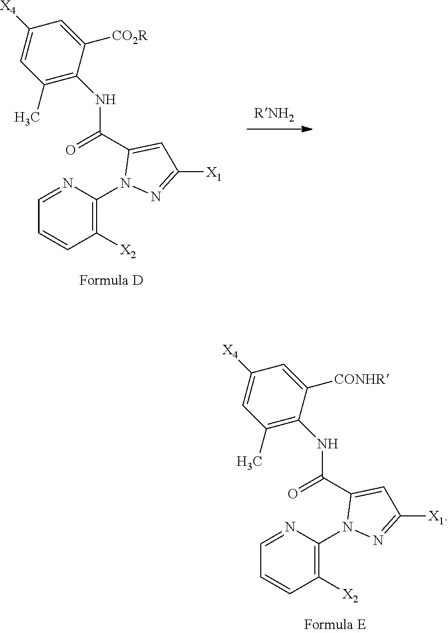

2. The process according to claim 1, wherein Formula E represent a compound wherein X₁=bromine, X₄=chlorine and R′=methyl.

3. The process according to claim 1, wherein Formula E represent a compound wherein X₁=bromine, X₂=chlorine, X₄=—CN and R′=methyl.

4. The process according to claim 1, wherein said halogenating agent is selected from thionyl chloride, phosgene, oxalyl chloride, phosphorus oxychloride, phosphorus trichloride, and phosphorus pentachloride.

5. The process according to claim 1, wherein said halogenated hydrocarbon solvent is selected from dichloromethane, dichloroethane, trichloromethane, chloroform, carbon tetrachloride, and a halogenated aromatic hydrocarbon.

6. The process as claimed in claim 1, wherein the process is carried out at temperature in the range from 30 to 70° C.

7. The process according to claim 1, wherein the compound of Formula A is 3-halo-1-(3-halo-2-pyridinyl)-4,5-dihydro-1H-pyrazole-5-carboxylic acid and the compound of formula of B is 3-halo-1-(3-halo-2-pyridinyl)-1H-pyrazole-5-carbonyl halide.

8. The process according to claim 1, wherein in the compound of Formula C X₄=chlorine and R=methyl.

9. The process according to claim 1, wherein in the compound of Formula C X₄=—CN and R=methyl.

10. A process for preparation of a compound of Formula B, comprising subjecting a compound of Formula A to pyrazoline aromatization by reacting with a halogenating agent in a halogenated hydrocarbon solvent wherein X₁, X₂ and X₃ are independently selected from bromine and chlorine

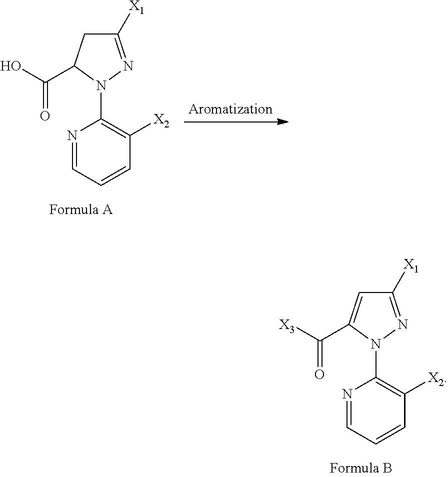

11. The process according to claim 10, wherein the compound of formula A is 3-halo-1-(3-halo-2-pyridinyl)-4,5-dihydro-1H-pyrazole-5-carboxylic acid and the compound of formula B is 3-halo-1-(3-halo-2-pyridinyl)-1H-pyrazole-5-carbonyl halide.

12. The process according to claim 10, wherein said process comprises preparation of a compound of Formula B1 (3-bromo-1-(3-chloro-2-pyridinyl)-1H-pyrazole-5-carbonyl chloride) by subjecting a compound of Formula A1 (3-bromo-1-(3-chloro-2-pyridinyl)-4,5-dihydro-1H-pyrazole-5-carboxylic acid) to pyrazoline aromatization by reacting the halogenating agent in the halogenated hydrocarbon solvent

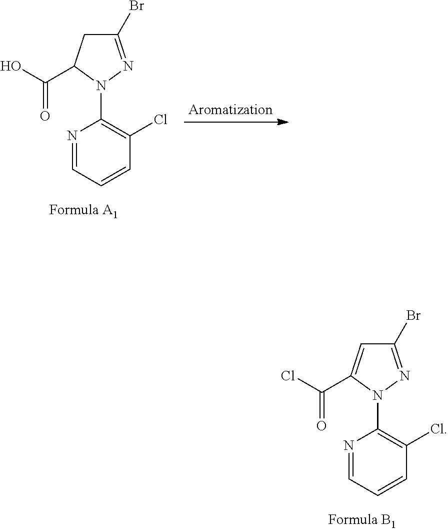 [SMILES: O=C(O)C1CC(Br)=NN1c1ncccc1Cl ; O=C(Cl)c1cc(Br)nn1-c1ncccc1Cl]

13. A compound of Formula B1 (3-bromo-1-(3-chloro-2-pyridinyl)-1H-pyrazole-5-carbonyl chloride) substantially free of a compound of formula B2, wherein Z is —OH or chlorine

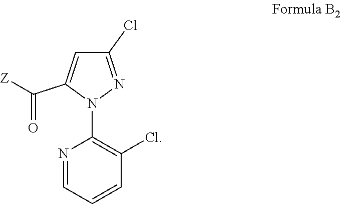

14. The compound of Formula B1 according to claim 13, containing less than 1.0% by weight of the compound of Formula B2, wherein Z is chlorine.

15. An anthranilamide compound of Formula E substantially free from a compound of formula B2, wherein Z is —OH or chlorine.

16. The anthranilamide compound of Formula E according to claim 15, containing less than 1.0% by weight of the compound of formula B2, wherein Z is chlorine.

17. The anthranilamide compound of Formula E according to claim 15, which is chlorantraniliprole or cyantraniliprole that is substantially free from compound of formula B2.

18. The anthranilamide compound of Formula E according to claim 17, containing less than 1.0% by weight a compound of formula E2.

19. The anthranilamide compound of Formula E according to claim 15, containing less than 1.0% by weight a compound of formula E4.

20. A process for the preparation of an anthranilamide compound of Formula E, comprising reacting a compound of Formula D with a compound of formula R′NH₂ wherein R′=a lower alkyl group or cycloalkyl, to form the anthranilamide compound of Formula E; wherein the reaction is carried out in a halogenated hydrocarbon solvent

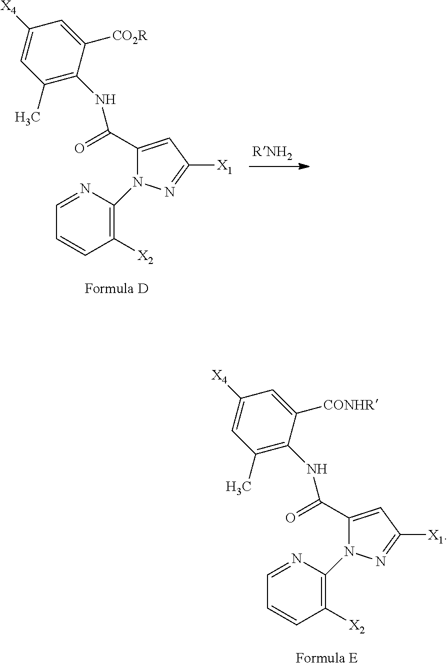

21. The process for the preparation of the anthranilamide compound of Formula E according to claim 20, wherein said halogenated hydrocarbon solvent is selected from dichloromethane, dichloroethane, trichloromethane, chloroform, carbon tetrachloride, and a halogenated aromatic hydrocarbon.

22. An insecticidal composition comprising chlorantraniliprole or cyantraniliprole produced according to process of claim 1 and an agrochemically acceptable excipient.

23. A method of controlling insects comprising applying to the insects or to their locus an insecticidally effective amount of chlorantraniliprole or cyantraniliprole produced according to the process of claim 1.
